(19) United States
(12) Patent Application Publication
(10) Pub. No.: US 2015/0004592 A1
(43) Pub. Date: Jan. 1, 2015

(54) SAMPLE STORAGE FOR MOLECULAR AND IMMUNOLOGICAL DETECTION

(71) Applicants: Alberto van Olphen, Tampa, FL (US); Maria Theresa Trindade, Tampa, FL (US); Cynthia Bucher, Temple Terrace, FL (US); David J. Matthes, St. Petersburg, FL (US)

(72) Inventors: Alberto van Olphen, Tampa, FL (US); Maria Theresa Trindade, Tampa, FL (US); Cynthia Bucher, Temple Terrace, FL (US); David J. Matthes, St. Petersburg, FL (US)

(73) Assignee: UNIVERSITY OF SOUTH FLORIDA, Tampa, FL (US)

(21) Appl. No.: 13/612,127

(22) Filed: Sep. 12, 2012

Related U.S. Application Data
(63) Continuation of application No. PCT/US2011/028325, filed on Mar. 14, 2011.
(60) Provisional application No. 61/313,296, filed on Mar. 12, 2010.

(51) Int. Cl. C12Q 1/70

(52) U.S. Cl. CPC C12Q 1/70; G01N 2333/11
USPC 435/5; 435/283.1

(57) ABSTRACT

A method for the direct molecular analysis of nucleic acid or antibodies from a natural or synthetic solid matrix for storing, preserving, and transporting nucleic acid or antibodies samples and simultaneously inactivating potential pathogens in the sample. A solid medium provides dry preservation of nucleic acid or antibodies, for example a filter paper treated with a novel chemical compound. This eliminates the tedious wash steps of conventional methods and allows for immediate processing of a clinical sample once it is received in the laboratory. This method is simple, rapid, and can be used to detect sensitive levels of pathogens in a complex matrix.

### CROSS-REFERENCE TO RELATED APPLICATION

This application is a continuation of and claims priority to International Patent Application No. PCT/US2011/028325 which was filed on Mar. 14, 2011, entitled “Sample Storage for Molecular and Immunological Detection”, which is a nonprovisional of and claims priority to U.S. Provisional Application No. 61/313,296 which was filed on Mar. 12, 2010, entitled “Simple and Rapid Method for Direct Molecular Detection of Nucleic Acid Samples from Filter Paper”. These patent applications are incorporated by reference into this disclosure.

### STATEMENT OF GOVERNMENT INTEREST

This invention was made with Government support under Grant No. W911SR-07-C-0084, awarded by Department of Defense. The government has certain rights in the invention.

### FIELD OF INVENTION

This invention relates to methods of detecting pathogens in clinical or research samples, detecting and preserving antibodies in samples, and preserving nucleic acids for archiving and detection.

### BACKGROUND OF INVENTION

There is an imperative need for a method to analyze directly clinical samples collected from patients who are potentially infected with a pathogen of serious concern. This will be especially true in developing regions of the world, where access to sophisticated laboratory techniques is limited, and most samples must be shipped long distances for analysis.

Currently, clinical samples are shipped in liquid media under refrigeration. Once received, the samples are processed and analyzed. A method enabling rapid sample testing is desirable and necessary, especially in the face of an epidemic.

Specifically, clinical specimens harboring potentially pathogenic viruses are collected with swabs, which are then deposited in sterile transport containers for shipment and subsequent laboratory analysis. These samples are maintained at 4° C. during transit and can be stored no longer than 4 days at this temperature. The process is time consuming, costly and impractical, especially in developing countries, where complicated protocols are difficult to follow.

### SUMMARY OF INVENTION

The present invention provides a method for the direct molecular analysis of nucleic acid and antibodies from a solid medium that can be used to store, preserve, and transport nucleic acid and protein samples, while simultaneously inactivating potential pathogens that may be present. The invention also provides a solid medium, comprising a solid absorbent matrix, natural or synthetic, for preserving nucleic acids and antibodies, and a solution of novel composition, into which the absorbent solid matrix is submerged prior to drying for storage and preservation.

This technology has been proven to eliminate the tedious wash steps of conventional approaches, and it allows for immediate processing of a clinical sample once received in the lab. The method is simple, rapid, and useful for sensitive detection of pathogens in a complex matrix.

### BRIEF DESCRIPTION OF THE DRAWINGS

For a fuller understanding of the invention, reference should be made to the following detailed description, taken in connection with the accompanying drawings, in which:

FIG. 1 is a depiction of the experimental detection of Influenza A Wyoming/03/2003 virus stored on FTA® cards for 1 hour.

FIG. 2 is a depiction of the experimental detection of Influenza A Wyoming/03/2003 virus stored on STM cards for 1 hour.

FIG. 3 is a depiction of the experimental detection of virus stored on STM cards stored for 3 weeks at room temperature (22±3° C.).

FIG. 4 is a depiction of 10 L of polyclonal antibody specific for the NS1 protein of Influenza A virus applied to an STM card.

FIG. 5 is a depiction of an analysis of preserved DNA on STM cards.

FIG. 6 is a depiction of an analysis by polymerase chain reaction (PCR) of clinical samples on FTA® cards and STM cards.

FIG. 7 is a depiction of real-time-PCR amplification curves for DNA of Patient 2 stored on FTA® cards and STM cards.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention.

In the broadest aspect, the present invention provides a method for the direct molecular analysis of nucleic acids and antibodies. The method comprises submerging an absorbent soled matrix into a solution of novel composition, drying the matrix at room temperature, and applying nucleic acids or antibodies to the matrix.

In another embodiment, The present invention provide a novel storage solution which when absorbed by a solid absorbent matrix, the solution consists essentially of a chelating agent and a buffering salt that has an ionization constant in the range pH 6-8. In a specific embodiment, the bromo-nitro-alkane-ol is 2-bromo-2-nitropropane-1,3-diol or its derivatives. The chelating agent is ethylene diamine tetracetic acid (EDTA) and the buffering salt is Tris.

The invention can be used to store, preserve, and transport nucleic acid or antibodies samples, and simultaneously inactivate potential pathogens in the sample. Samples prepared by the method of the invention can be used for subsequent molecular analysis and/or serological evaluation. Samples can also be shipped safely dry and at ambient temperature.

The invention can be used to preserve and transport clinical or research samples containing human or animal viruses from the Adenoviridae, Papillomaviridae, Parvoviridae, Herpesviridae, Poxviridae, Hepadnaviridae, Polyomaviridae, Circoviridae, Reoviridae, Picornaviridae, Caliciviridae, Togaviridae, Arenaviridae, Flaviviridae, Orthomyxoviridae, Paramyxoviridae, Bunyaviridae, Rhabdoviridae, Filoviridae, Coronaviridae, Astroviridae, Bornaviridae and Retroviridae families, or plant viruses.

In a preferred embodiment of the invention, sterile filter paper is used as the solid absorbent matrix. STM cards are made by using sterile filter paper saturated in a novel solution containing a 1:300 dilution of 2-bromo-2-nitropropane-1,3-diol, 10 mM EDTA, and 60 mM tris buffer. The filter paper is soaked in the solution and allowed to dry at room temperature. As a specific example, serial dilutions of a virus sample are prepared. 5 L of each concentration are then applied to the filter paper and allowed to dry for 1 hour at 25° C. The samples are then tested for nucleic acid preservation. Circular pieces, 2 mm in diameter, one for each concentration, are then removed from the filter paper and placed directly into a plastic tube for molecular analysis. Real-time-PCR analysis is then done directly from the filter paper samples. No washing of samples between collection and analysis by real-time PCR is required.

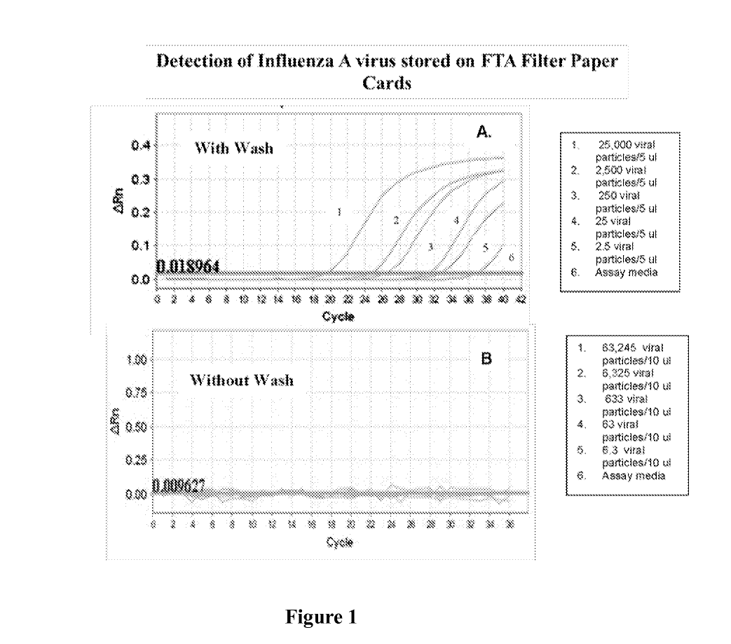

A comparison has been made between FTA® card samples which were rinsed or not rinsed for direct molecular analysis by real time-PCR. FIG. 1 shows the need for intermediate wash steps between sample collection and genetic analysis of a sample. In FIG. 1A, four wash steps were done with the FTA® reagent in TE buffer; the incubation time for each wash was 5 min. Virus was detected in 25 samples. In FIG. 1B, Influenza A Wyoming/03/2003 virus was stored on FTA® cards for 1 hour. These samples were not rinsed. Virus was not detected in any sample. The data show that rinsing steps are necessary for samples stored on FTA® cards.

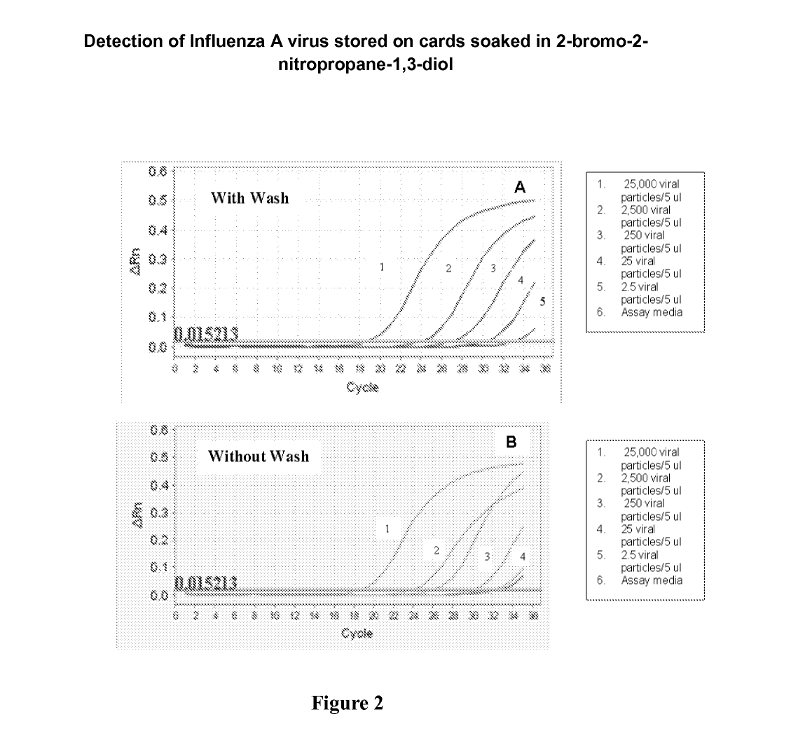

The same comparison has been made with samples deposited on filter paper containing the novel 2-bromo-2-nitropropane-1,3-diol formulation. FIG. 2 shows that Influenza A virus can be detected at sensitive levels directly from the STM cards in the absence of intermediate wash steps. In FIG. 2A, two wash steps were done with SDS buffer; the incubation time was 5 min per rinse. Virus was detected in 25 samples. In FIG. 2B, Influenza A Wyoming/03/2003 virus was stored on STM cards and analyzed using real time PCR. Even though samples were not washed, viral nucleic acid was detected at the same level (25 viral samples) than the control that included a wash step (FIG. 2A), indicating that the STM cards, in contrast to FTA® cards (FIG. 1B), adds advantage of detecting nucleic acid without the need for tedious washes/rinses.

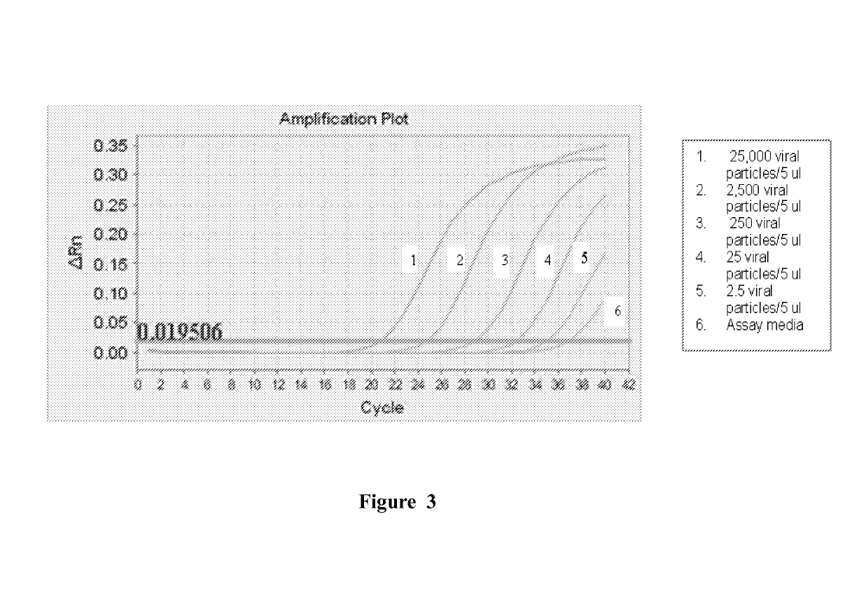

Further studies with STM cards showed that this technology can preserve nucleic acids after 17 days of incubation at 25° C. without loss of sensitivity. FIG. 3 shows that there was no loss of RNA detection sensitivity after 3 weeks of storage of virus on STM cards

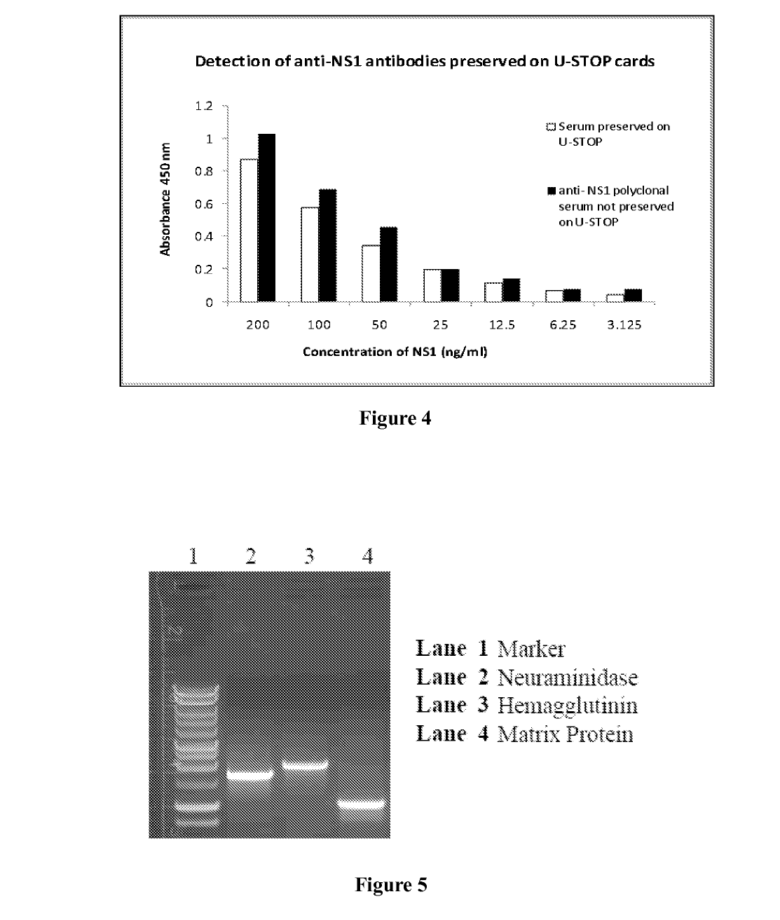

FIG. 4 presents data on antibody preservation and detection on STM cards. 10 L of polyclonal antibody specific for the NS1 protein of Influenza A virus was applied to an STM card. The serum was allowed to dry on the card at 25° C. Antibody was eluted off the card in PBS overnight at 4° C. An ELISA was done to assess the ability of the STM card to preserve an antibody sample for future serological testing. Fresh samples and preserved samples were comparable in affinity for NS1. Storing virus samples on STM cards resulted in only limited loss of sensitivity.

FIG. 5 demonstrates that the STM cards are effective in preserving the integrity of larger nucleic acid fragments allowing the analysis of RNA on STM cards containing influenza virus. Specific primers for the hemagglutinin gene, neuraminidase gene and matrix gene of influenza virus were used for amplification by PCR directly from the STM cards containing influenza virus and the products were analyzed by gel electrophoresis.

The foregoing data show that the method of the present invention represents clear advantages over an existing commercial method (U.S. Pat. No. 5,807,527) for the preservation and transport of viruses. The present invention saves time, eliminates intermediate wash steps between sample collection and molecular analysis, and reduces the likelihood of cross-contamination between samples.

### EXAMPLE

A comparison has been made between clinical samples stored on FTA® cards and on 2-bromo-2-nitropropane-1,3-diol-pretreated STM cards. The stored cards were compared with regard to their ability to preserve nucleic acid from influenza virus for detection by reverse transcriptase-real time-PCR. Nasal swabs from patients suspected of having influenza virus infection were placed into M4 transport medium. 50 L aliquots of the resulting samples were applied directly to an FTA® card or an STM card. The cards were allowed to dry at room temperature before storage. A second set of cards was prepared as follows. The clinical samples in M4 medium were centrifuged and the pellets were resuspended in small volumes. 50 L aliquots of the pellets were applied to an FTA® card or an STM card. These cards too were allowed to dry at room temperature. The cards were mailed to the University of South Florida virology core laboratory for testing by real time-PCR. A 2 mm circular sample was obtained from each STM card and transferred to a PCR tube for testing. There was no wash step. The FTA® cards were washed twice with 100 L of FTA® reagent and then twice with 0.01% sodium dodecyl sulfate, per the manufacturer's recommendation. A 2 mm sample was obtained from each card and transferred to a PCR tube for testing. A blank filter paper was punched between independent samples to avoid cross contamination between STM card samples and FTA® card samples.

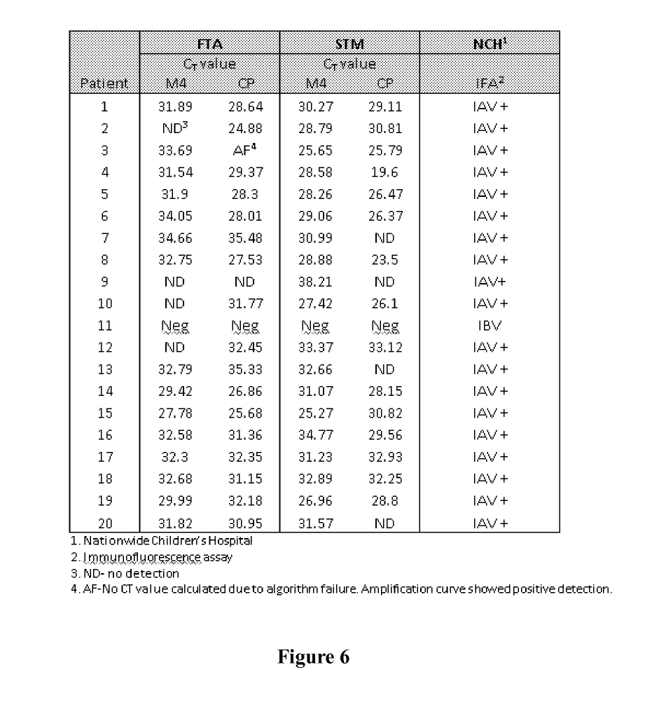

The clinical samples of Paragraph [0034] were independently tested for Influenza A and B by a direct immunofluorescence (DFA) test in Nationwide Children's Hospital. All the samples tested positive for Influenza A virus by reverse transcriptase-PCR except Sample 11, which was positive for Influenza B virus. FIG. 6 shows the results of the analysis.

The data show that the quality of samples stored on STM cards compared favorably with the quality of samples stored on FTA® cards, even though the STM cards were processed directly, that is, without the wash steps required for analysis of samples on the FTA® cards. The preparation time for the clinical samples was seconds for the STM cards, whereas the FTA® cards required 20 min of washing before the PCR assay was performed. The samples from both card types were tested 2 weeks after collection. Eight of the 20 samples tested generated lower CT values from the STM cards, indicating that the nucleic acid may be more stable on these cards than on the FTA® cards. Results for all other samples on the STM cards were comparable to those obtained with the FTA® cards.

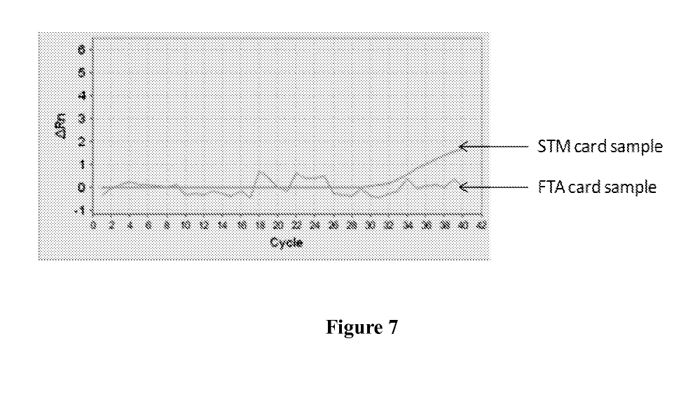

It is also shown that the FTA® card samples also generated erratic background readings likely caused by the residue left on the card from the wash steps after drying. FIG. 7 shows amplification curve results for patient 2 on both types of cards with real time RT-PCR. Both samples were from the M4 medium in which the swabs were placed after the sample was taken. The inconsistent fluorescence readings could affect the CT value calculated by the PCR instrument and ultimately whether or not the sample is designated as positive or negative for the virus tested.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall there between.

What is claimed is:

1. A solid medium for storing, preserving and transporting clinical or research samples of nucleic acids or antibodies, the solid medium comprising:
A natural or synthetic solid absorbent matrix;
An effective amount of preservation solution to protect against degradation of nucleic acids or antibodies adsorbed onto the solid absorbent matrix, said composition comprising:
A bromo-nitro-alkane-ol;
A chelating agent;
A buffering salt that has an ionization constant in the range pH 6-8.

2. The solid medium according to claim 1 wherein the bromo-nitro-alkane-ol is 2-bromo-2-nitropropane-1,3-diol or its derivatives.

3. The solid medium according to claim 1 wherein the solid matrix is filter paper.

4. The solid medium according to claim 1 wherein the chelating agent is EDTA.

5. The solid medium according to claim 1 wherein the buffering salt is Tris.

6. The solid medium according to claim 1 wherein the preservation solution is absorbed by the solid absorbent matrix before adsorption of a sample of nucleic acid or antibodies.

7. The solid medium according to claim 1 wherein the nucleic acid or antibodies stored and retrived from the solid medium is subsequently analyzed.

8. A storage solution which when absorbed by a solid absorbent matrix, providing a dry solid medium for storage and presavation of sample nucleic acid or antibodies, the solution consisting of a bromo-nitro-alkane-ol, a chelating agent and a buffering salt that has an ionization constant in the range pH 6-8.

9. The storage solution according to claim 8 wherein the bromo-nitro-alkane-ol is 2-bromo-2-nitropropane-1,3-diol or its derivatives.

10. A method for storage of a sample of nucleic acid or antibodies, the method comprising:
providing a dry solid medium comprising a natural or synthetic solid absorbent matrix and a composition consisting essentially of a bromo-nitro-alkane-ol, a chelating agent and a buffering salt that has an ionization constant in the range pH 6-8, wherein the composition is incorporated into the solid matrix; and
allowing the solid medium to dry at room temperature;
applying the nucleic acid or antibody sample to the dry solid medium;
storing the sample applied to the dry solid medium.

11. The method for storage according to claim 10 wherein the bromo-nitro-alkane-ol is 2-bromo-2-nitropropane-1,3-diol or its derivatives.
